(19) United States
(12) Plant Patent Application Publication
(10) Pub. No.: US 2022/0330466 P1
(43) Pub. Date: Oct. 13, 2022

(54) CANNABIS PLANT NAMED 'VB252X'

(50) ;

(71) Applicant: Verda Bio, LLC, Seattle, WA (US)

(72) Inventors: JESSICA TONANI, SEATTLE, WA (US); KERSTEN GABA, SEATTLE, WA (US)

(21) Appl. No.: 17/658,570

(22) Filed: Apr. 8, 2022

Related U.S. Application Data
(60) Provisional application No. 63/172,432, filed on Apr. 8, 2021.
(60) Provisional application No. 63/174,870, filed on Apr. 14, 2021.

(51) Int. Cl. A01H 6/28; A01H 5/12

(52) U.S. Cl. CPC A01H 6/28; A01H 5/12
USPC 800/298

(57) ABSTRACT

The unique annual herbaceous Cannabis plant variety ‘VB252X’ is provided. The variety can be distinguished by its outstanding features of increased vigor and large size.

### CROSS REFERENCE TO RELATED APPLICATIONS

The present Application for Patent claims priority to Provisional Application No. 63/172,432 entitled “CANNABIS PLANT NAMED ‘VB252’” filed Apr. 8, 2021, and Provisional Application No. 63/174,870 entitled “CANNABIS PLANT NAMED ‘VB252X252’” filed Apr. 22, 2021, which are hereby expressly incorporated by reference herein.

Latin name of the genus and species: Genus—Cannabis. Species—sativa.

Variety denomination: The new Cannabis plant claimed is of the variety denominated ‘VB252X’.

### BACKGROUND OF THE INVENTION

### Field of the Invention

The present invention relates to a new and distinct annual variety of C. sativa, which has been given the variety denomination of ‘VB252X’. ‘VB252X’ is intended for use in the production of products created from the leaf, stem or flowers of the plant including products created from extracting the oil from the plant. The oil products can include, but are not limited to, production of cannabinoid oil formulations to be used in consumer product goods, medicinal formulation to be sold in dispensaries, pharmaceutical oil formulations, nutraceutical formulation and other consumer product

### Background Of The Related Art

The genus Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense legal commercialization in the United States as industrial hemp for a variety of purposes including biodegradable plastics and building materials, clothing, paper, food, fuel, and medicines.

Cannabidiol (CBD) extracted from Cannabis is widely used in over-the-counter medicines and topical treatments and is also the active ingredient in the FDA-approved drug Epidiolex®. CBD is just one of at least dozens—perhaps hundreds—of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1093/pm/pnz227; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1(1): 154-165, each of which is herein incorporated by reference in its entirety for all purposes.

Non-hemp forms of Cannabis, frequently referred to as marijuana, have been legalized for medicinal use in many states and also for adult use in a growing number of states. It is expected that the wave of legalization will continue to the point of some form of federal legalization or decriminalization.

Typically, marijuana products are available to users for purchase in specialized “dispensaries” that offer dried flower, edibles, tinctures, extracts, and the like. In some cases, a unique or unusual chemical profile, or chemotype, is attractive not only for flower sales but also for use in the preparation of extracts and/or isolates and for the manufacture of a variety of products that possess characteristics of the chemotype.

### SUMMARY OF THE INVENTION

The present invention relates to a new and distinct annual variety of C. sativa, which has been given the variety denomination of ‘VB252X’. ‘VB252X’ is intended for use in the production of products created from the leaf, stem, or flowers of the plant, including products created from extracting the oil from the plant. These oil products can include, but are not limited to, production of cannabinoid oil formulations to be used in consumer product goods, medicinal formulation to be sold in dispensaries, pharmaceutical oil formulations, nutraceutical formulation, and other consumer products.

The new C. sativa variety is a selection resulting from a sexual cross of C. sativa plants at Seattle, Wash., involving a seed parent known as ‘VB81’ (not patented) and a pollen parent known as ‘VB214’ (not patented).

Selection was based upon agronomic characteristics, general phenotypic appearance, and oil production attributes. Analysis for traits was done using HPLC and TLC for compound analysis. Growth performance was evaluated in growth pods, greenhouse, and outdoor growing conditions.

‘VB252X’ was created from multiple generations of inbreeding. An initial F1 cross was completed on seeds with complementary traits. Two siblings from the F1 cross were selected due to agronomical traits and crossed (F2, S1). From the F2 cross, a single plant was selected for its agronomical traits and was backcrossed to the original F1 pollen donor (BC1, F3). Single plant selection was completed, and the top female plant was carried forward and backcrossed once again to the original pollen donor (BC2, F4). A single plant selection was completed for the F4 cross, and the top female plant was carried forward and back-crossed once again to the original pollen donor (BC3, F5). VB252X was selected from that last cross. Selection was based upon agronomic characteristics, general phenotypic appearance, and oil production attributes. VB252X was selected from that last cross.

The selection was subsequently evaluated for 3 years at Seattle, Wash. and the Yakima Valley, Wash.

The following characteristics of the new variety have been repeatedly observed and can be used to distinguish ‘VB252X’ as a new and distinct variety of C. sativa plant:
1. Phenotypically, ‘VB252X’ have physical characteristics of a Cannabis hybrid, i.e., plants have physical characteristics of both C. sativa and C. indica.
2. It is a photoperiod-sensitive variety with a later flowering time. Harvest is approximately 7 weeks after induction.
3. Leaves are medium green and, phenotypically, would be characterized as C. indica (i.e., broad large leaf structures).
4. It has short internode spacing with full branching.
5. Branches contain multiple nodes per branch.
6. It is a larger than most C. sativa plants (favoring a C. indica phenotype) having a propensity to grow over 6 feet tall within a standard 3-4-month outdoor grow season.
7. ‘VB252X’ is a robust growing plant.

Plants of the new variety differ from plants of the seed parent ‘VB81’ primarily in growth and phytochemical content. ‘VB252X’ is a more robust plant than ‘VB81’. ‘VB252X’ has increased vigor and thrives in multiple growing conditions in contrast to ‘VB81’, which only thrives in indoor growing pods. ‘VB81’ is spindly and is highly susceptible to pathogens. ‘VB252X’ is less susceptible to pathogen. ‘VB252X’ grows larger with broader leaves than ‘VB81’. Phytochemically, both ‘VB81’ and ‘VB252X’ produce high levels of CBD and contain similar phytochemical profiles. ‘VB252X’ produces larger flowers and increased production over ‘VB81’.

Plants of the new variety differ from plants of the pollen parent ‘VB214’ primarily in growth and phytochemical content. ‘VB214’ is male plant and does not produce flowers. The limited trichomes on the leaves produce predominantly CBD, but due to low chemical production in males, it is difficult to do an exact comparison of cannabinoid predisposition between the two plants. ‘VB214’ is a robust growing plant (attributes to ‘VB252X’ plant vigor). It is robust and resistant to many pathogens common in Cannabis including many forms of powdery mildew and some common pest infestation (e.g., mites). Like many male pollen plants, ‘VB214’ has less branching than ‘VB252X’ (longer spacing), is taller, and contains fewer leaves.

Asexual reproduction of the new variety has demonstrated that the new variety reproduces true to type with all of the characteristics, as herein described, firmly fixed and retained through successive generations of such asexual propagation.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying photographic illustrations show the typical appearance of the new variety ‘VB252X’. The colors are as nearly true as is reasonably possible in a color representation of this type. Colors in the photographs may differ slightly from the color values cited in the detailed botanical description which accurately describes the colors of the new plant.

FIG. 1 is a photograph of the whole plant of the new variety ‘VB252X’.

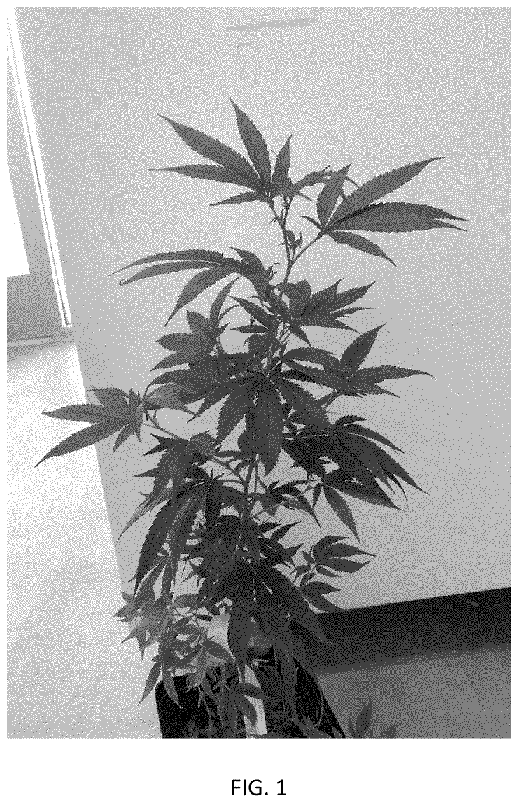

FIG. 2 is a photograph of the inflorescence of new variety ‘VB252X’.

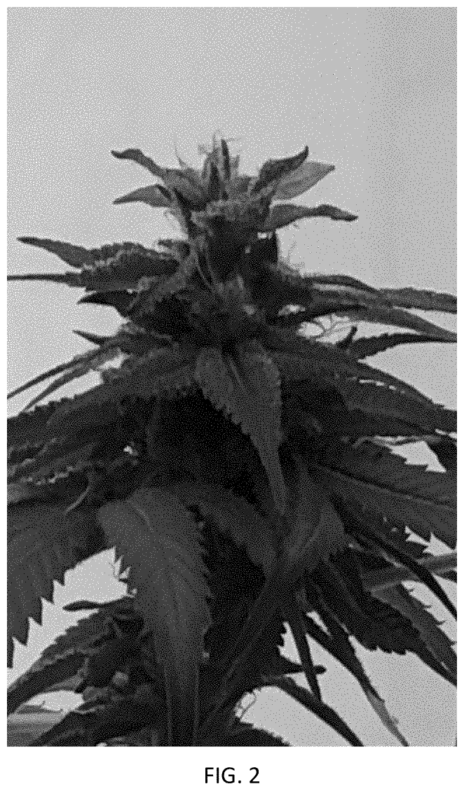

### DETAILED DESCRIPTION

Some embodiments of the invention relate to a seed from a Cannabis plant designated ‘VB252X’ wherein a representative sample of seed of said plant has been deposited under ______.

Some embodiments of the invention relate to a Cannabis plant, or plant part, tissue, or cell thereof produced by growing the seed of ‘VB252X’, or a descendant thereof. Plant parts can include the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like.

The plants, or plant parts, of the invention can display a cannabinoid profile within the ranges set forth in Table 1 herein. The productivity of any given cannabinoid and/or the amounts or ratios of cannabinoids, terpenes, and other plant products can be, by nature, quite variable. The variability can be contributed to by weather, latitude, soil and feeding conditions, pathogens, and numerous other agronomic, horticultural, and biological factors.

Some embodiments of the invention relate to methods of using the plant in a breeding program to produce Cannabis progeny including a cannabinoid profile generally within the ranges as set forth in Table 1 herein. Details of existing Cannabis plant varieties and breeding are described in Potter et al. (2011, World Wide Weed: Global Trends in Cannabis Cultivation and Its Control); Holland (2010, The Pot Book: A Complete Guide to Cannabis, Inner Traditions/Bear & Co, ISBN1594778981, 9781594 778988); Green I (2009, The Cannabis Grow Bible: The Definitive Guide to Growing Marijuana for Recreational and Medical Use, Green Candy Press, 2009, ISBN 1931160589, 9781931160582); Green II (2005, The Cannabis Breeder's Bible: The Definitive Guide to Marijuana Genetics, Cannabis Botany and Creating Strains for the Seed Market, Green Candy Press, 1931160279, 9781931160278); Starks (1990, Marijuana Chemistry Genetics, Processing & Potency, ISBN 0914171399, 9780914171393); Clarke (1981, Marijuana Botany, an Advanced Study: The Propagation and Breeding of Distinctive Cannabis, Ronin Publishing, ISBN 091417178X, 9780914171782); Short (2004, Cultivating Exceptional Cannabis: An Expert Breeder Shares His Secrets, ISBN 1936807122, 9781936807123); Cervantes (2004, Marijuana Horticulture: The Indoor/Outdoor Medical Grower's Bible, Van Patten Publishing, ISBN 187882323X, 9781878823236); Franck et al. (1990, Marijuana Grower's Guide, Red Eye Press, ISBN 0929349016, 9780929349015); Grotenhermen and Russo (2002, Cannabis and Cannabinoids: Pharmacology, Toxicology, and Therapeutic Potential, Psychology Press, ISBN 0789015080, 9780789015082); Rosenthal (2007, The Big Book of Buds: More Marijuana Varieties from the World's Great Seed Breeders, ISBN 1936807068, 9781936807062); Clarke, R C (Cannabis: Evolution and Ethnobotany 2013); King, J (Cannabible Vols 1-3, 2001-2006); and four volumes of Rosenthal's Big Book of Buds series (2001, 2004, 2007, and 2011), each of which is herein incorporated by reference in its entirety for all purposes.

The present invention also relates to variants, mutants and minor modifications of the seeds, plant parts and/or whole plants of the Cannabis plants of the present invention. Variants, mutants and minor modifications of the seeds, plants, plant parts, plant cells of the present invention can be generated by methods well known and available to one skilled in the art, including but not limited to, mutagenesis (e.g., chemical mutagenesis, radiation mutagenesis, transposon mutagenesis, insertional mutagenesis, signature tagged mutagenesis, site-directed mutagenesis, and natural mutagenesis), knock-outs/knock-ins, antisense and RNA interference. For more information of mutagenesis in plants, such as agents, protocols, see Acquaah et al. (Principles of plant genetics and breeding, Wiley-Blackwell, 2007, ISBN 1405136464, 9781405136464,) which is herein incorporated by reference in its entirety. Other kinds of modifications practiced in the Cannabis industry, including but not limited to feminization of seeds and/or day-length neutrality/autoflowering are also within the scope of the invention and are within the level of skill in the art to execute.

The present invention also relates to a mutagenized population of the Cannabis plants of the present invention, and methods of using such populations. In some embodiments, the mutagenized population can be used in screening for new Cannabis lines which comprises one or more or all of the morphological, physiological, biological, and/or chemical characteristics of Cannabis plants of the present invention.

In some embodiments, the new Cannabis plants obtained from the screening process comprise one or more or all of the morphological, physiological, biological, and/or chemical characteristics of Cannabis plants of the present invention, and one or more additional or different new morphological, physiological, biological, and/or chemical characteristic.

The present invention also provides any compositions or any products made from or isolated from the plants of the present invention. In some embodiments, the compositions/ products comprise an extract of the plants. In some embodiments, the extract can contain a higher percentage of terpenes/terpenoids compared to extract isolated from a control Cannabis plant variety (e.g., an existing variety, such as a recreational Cannabis plant variety). In some embodiments, the invention relates to a smokable, topical, or edible product comprising the Cannabis plant, or plant part, tissue, cell, extract, or isolate.

The present invention provides methods of using the Cannabis plants or any parts, any compositions, or any chemicals derived from said plants of the present invention.

In some embodiments, the plants of the present invention can be used to produce new plant varieties. In some embodiments, the plants are used to develop new varieties or hybrids with desired phenotypes or genotypes.

In some embodiments, selection methods, e.g., molecular marker assisted selection, can be combined with breeding methods to accelerate the process. Additional breeding methods known to those of ordinary skill in the art include, e.g., methods discussed in Chahal and Gosal (Principles and procedures of plant breeding: biotechnological and conventional approaches, CRC Press, 2002, ISBN 084931321X, 9780849313219); Taji et al. (In vitro plant breeding, Routledge, 2002, ISBN 156022908X, 9781560229087); Richards (Plant breeding systems, Taylor & Francis US, 1997, ISBN 0412574500, 9780412574504); Hayes (Methods of Plant Breeding, Publisher: READ BOOKS, 2007, ISBN1406737062, 9781406737066); each of which is incorporated by reference in its entirety. The Cannabis genome has been sequenced (Bakel et al., The draft genome and transcriptome of Cannabis sativa, Genome Biology, 12(10):R102, 2011). Molecular makers for Cannabis plants are described in Datwyler et al. (Genetic variation in hemp and marijuana (Cannabis sativa L.) according to amplified fragment length polymorphisms, J Forensic Sci. 2006 March; 51(2):371-5.); Pinarkara et al., (RAPD analysis of seized marijuana (Cannabis sativa L.) in Turkey, Electronic Journal of Biotechnology, 12(1), 2009), Hakki et al., (Inter simple sequence repeats separate efficiently hemp from marijuana (Cannabis sativa L.), Electronic Journal of Biotechnology, 10(4), 2007); Gilmore et al. (Isolation of microsatellite markers in Cannabis sativa L. (marijuana), Molecular Ecology Notes, 3(1): 105-107, March 2003); Pacifico et al., (Genetics and marker assisted selection of chemotype in Cannabis sativa L.), Molecular Breeding (2006) 17:257-268); and Mendoza et al., (Genetic individualization of Cannabis sativa by a short tandem repeat multiplex system, Anal Bioanal Chem (2009) 393:719-726); each of which is herein incorporated by reference in its entirety.

In some embodiments, the Cannabis plant, or plant part, tissue, or cell of ‘VB252X’ comprises a cannabinoid profile as set forth in Table 1. Due to the natural variability of chemotypic expression that is commonly observed in Cannabis plants, arising from numerous causes as discussed above, the values set forth in Table 1 do not reflect the only possible range of outcomes that can be obtained from plants of the new variety. Thus, these values are merely exemplary of observed values (middle column) and predicted normal variations from the observed values. Variations outside these ranges are also within the scope of the invention.

TABLE 1
Exemplary Profiles of Key Cannabinoids.*

| Cannabinoid | Percent | Percent | Percent | Percent | Percent |
|---|---|---|---|---|---|
| THCa | 0 | 0.015 | 0.03 | 0.06 | 0.075 |
| Delta 9 THC | 0 | 0.0525 | 0.105 | 0.21 | 0.2625 |
| THCVa | 0 | 0.005 | 0.01 | 0.02 | 0.025 |
| THCV | 0 | 0.01 | 0.02 | 0.04 | 0.05 |
| CBDa | 3.225 | 4.3 | 4.485 | 4.67 | 5.8375 |
| CBD | 0.15 | 0.2 | 1.535 | 2.87 | 3.5875 |
| CBDVa | 0.375 | 0.5 | 0.585 | 0.67 | 0.8375 |
| CBDV | 0 | 0.1075 | 0.215 | 0.43 | 0.5375 |
| CBGa | 0.075 | 0.1 | 0.105 | 0.11 | 0.1375 |
| CBG | 0 | 0.02 | 0.04 | 0.08 | 0.1 |
| CBCa | 0.15 | 0.2 | 0.265 | 0.33 | 0.4125 |
| CBC | 0 | 0.0075 | 0.015 | 0.03 | 0.0375 |
| Total THC** | 0 | 0.065 | 0.13 | 0.26 | 0.325 |
| Total CBD*** | 3.075 | 4.1 | 5.53 | 6.96 | 8.7 |
| Total Cannabinoids**** | 4.125 | 5.5 | 7.51 | 9.52 | 11.9 |

*In some cases, the quantification of a component is reported as “not detected,” or ND, rather titan 0. For purposes of simplicity, ND is replaced with 0.
**Total THC = Δ9THC + (THCa * 0.877)
***Total CBD = CBD + (CBDa * 0.877)
****Total Cannabinoids = Total THC + Total CBD + Total CBG + Total THCV + Total CBC + Total CBDV + Δ8THC + CBL + CBN

In some embodiments, the invention relates to a Cannabis clone regenerated from the Cannabis plant of descended from the plant, or plant part, tissue, cell, or seed of ‘VB252X’ wherein the plant is a clonal descendent.

In some embodiments, the invention relates to a method of producing an F1 Cannabis seed, wherein the method includes crossing the plant with a different Cannabis plant and harvesting the resultant F1 Cannabis seed. In some embodiments, the invention relates to the F1 hybrid Cannabis seed produced by this method. In some embodiments, the invention relates to a F1 hybrid Cannabis plant produced by growing the F1 hybrid Cannabis seed. In some embodiments, the invention relates to a Cannabis clone regenerated from the F1 hybrid Cannabis plant. In some embodiments, the invention relates to a smokable or edible product comprising Cannabis tissue from the F1 hybrid Cannabis plant.

In some embodiments the invention relates to seed line from a clonally propagated plant of the new variety. In some embodiments, the seed line is that of the deposited seed recited herein. In other embodiments, the seed line is one that is separately established through interbreeding and selection of plants of the new variety, using pollen from reversed females of the new variety and/or from relatives/ancestors of the new variety. In these embodiments, crosses and selections are conducted through successive generations to obtain a line of seed that stably produces progeny having physical and chemical properties within the ranges recited herein for the new variety. In some embodiments, this seed line is feminized seed, having been feminized using techniques known to those of skill in the art.

The following detailed description sets forth the distinctive characteristics of ‘VB252X’. Applicant is prepared to make a deposit of seeds or plant tissue.

### DETAILED BOTANICAL DESCRIPTION

The following detailed description sets forth the distinctive characteristics of ‘VB252X’. The data which defines these characteristics was collected from asexual reproductions of the original selection. Dimensions, sizes, colors, and other characteristics are approximations and averages set forth as accurately as possible.

The plant history was taken on plants approximately <1 years of age, and the descriptions relate to plants grown in a greenhouse.
Type: Herbaceous tap-rooted annual.
   Classification.—a. Family— Cannabaceae. b. Genus—Cannabis. c. Species—sativa. d. Common name—Hemp.
Plant:
   General.—a. Origin— Seattle, Wash. b. Parentage: i. Male parent— ‘VB214’. ii. Female parent— ‘VB81’. c. Average height— Outdoor 6 feet/Indoor dependent on photoperiod length. d. Average spread— Outdoor 3 feet/Indoor dependent on photoperiod length. e. Productivity— Average outdoor 2 lbs. dried biomass. f. Proportion of hermaphrodite plants— Low. g. Proportion of female plants— High. h. Proportion of male plants— Low. i. Photoperiodicity— Photoperiod-sensitive. j. Plant Branching— Strong. k. Plant Height (At Flowering)— Tall (200 cm). l. Time of Flowering— 120 days after seeding.
Stem/central stalk:
   General.—a. Thickness— Medium. b. Average length of internode— Medium. c. Depth of grooves— Medium. d. Pith in cross section— Thick. e. Main Stem Color— Dark green. f. Plant Anthocyanin Coloration of Crown— Medium.
Leaf:
   General.—a. Leaf Intensity of Green Color— Medium. b. Number of leaflets per leaf— Medium (Majority=7). c. Central leaflet length— Medium (7.5 cm). d. Central leaflet width— Broad (2.5 cm).
Petiole:
   General.—a. Length— Medium (5 cm). b. Anthocyanin color intensity— Medium. c. Color— Medium.
Seeds:
   General.—Shape— Ovate/Oblong. Weight per 1000 seeds— 10.5 grams. Color of testa— Grey Brown (5YR 3/3; Munsell Soil Color Code). Marbling— Medium.

### Note

Applicant is prepared to submit a seed and/or tissue deposit of the variety herein described, prior to issuance or publication, as required by the law of the relevant jurisdiction, as needed to support claims reciting such a deposit.

What is claimed is:

1. A seed from a Cannabis plant designated ‘VB252X’ wherein a representative sample of seed of said plant has been deposited under ______.

2. A Cannabis plant, or plant part, tissue, or cell thereof produced by growing the seed of claim 1, or a descendant thereof.

3. The Cannabis plant, or plant part, tissue, or cell thereof of claim 2 comprising a cannabinoid profile set forth in Table 1.

4. Use of the plant of claim 2 in a breeding program to produce Cannabis progeny comprising a cannabinoid profile set forth in Table 1 and genetic capacity to produce the cannabinoid profile set forth in Table 1 in progeny thereof.

5. The Cannabis plant part of claim 2, wherein said plant part is selected from the group consisting of: stems, trichomes, leaves, and flower buds.

6. The Cannabis plant descended from the plant, or plant part, tissue, cell, or seed of claim 2, wherein the plant is a clonal descendent.
